(19) United States
(12) Patent Application Publication
(10) Pub. No.: US 2017/0322197 A1
(43) Pub. Date: Nov. 9, 2017

(54) Health Monitoring Toilet System

(71) Applicants: David R. Hall, Provo, UT (US); Joshua Larsen, Spanish Fork, UT (US); Jared Reynolds, Pleasant Grove, UT (US); Stephen C. Davis, Salt Lake City, UT (US); Joe Fox, Spanish Fork, UT (US)

(72) Inventors: David R. Hall, Provo, UT (US); Joshua Larsen, Spanish Fork, UT (US); Jared Reynolds, Pleasant Grove, UT (US); Stephen C. Davis, Salt Lake City, UT (US); Joe Fox, Spanish Fork, UT (US)

(21) Appl. No.: 15/652,727

(22) Filed: Jul. 18, 2017

Related U.S. Application Data
(63) Continuation-in-part of application No. 14/702,723, filed on May 2, 2015.

(51) Int. Cl. G01N 33/493; E03D 11/11; A61B 10/00; G01N 21/27; G01N 21/25; A61B 5/1172

(52) U.S. Cl. CPC G01N 33/493; G01N 21/27; G01N 21/255; E03D 11/11; A61B 10/007; A61B 5/1172

(57) ABSTRACT

A health monitoring toilet system collects urine in a toilet system which may resemble a traditional water toilet. The toilet system may include a urine capture basin for collecting urine as a user urinates as with a traditional toilet. The urine capture basin may include a urine entry aperture which is an orifice leading into a urine sample cell. A fiber optic spectrometer may analyze the urine in the urine sample cell to collect relevant health data. The system may include a controller which may store and analyze spectral data and compile longitudinal data over time. The system may be in connection with other such systems to compare the analysis of a user's urine with those of other users stored in other such health monitoring toilet systems.

### CROSS REFERENCE TO RELATED APPLICATIONS

This application is a continuation-in-part of co-pending U.S. patent application Ser. No. 14/702,723 filed on May 2, 2015 which is hereby incorporated by reference in its entirety.

### FIELD OF THE INVENTION

This disclosure relates to devices and methods of detecting urine analytes and managing the collected data to assess a user's health status.

### BACKGROUND OF THE INVENTION

Quantification of urine analytes may be used to assess health and diagnose disease. Although urine is a continuous and compulsory source of information pertaining to an individual's health, urine is typically tested only intermittently due to the practicality of collecting and testing urine. Consequently, an immense source of readily available health data is lost and healthcare providers are left with merely a snapshot of biological events which occur over time.

In an effort to compensate for the lack of longitudinal trend data, healthcare providers compare test results with population statistics. This provides some insight into population-relative health at a single point in time but provides little true insight into dynamic health processes of the individual.

Accordingly, there is a need to develop a biological waste product analysis system that improves on existing methods by generating longitudinal data from frequently acquired samples.

### BRIEF SUMMARY OF THE INVENTION

We disclose a health monitoring toilet system which collects urine from a user as the user urinates as one would into a traditional water toilet. The health monitoring toilet system may include a toilet bowl and a urine capture basin within the toilet bowl. The urine capture basin may include a urine entrance aperture which leads into a urine sample cell. The urine sample cell may act as a sample cell for a fiber optic spectrometer which may be included in the health monitoring toilet system.

A thermal sensor may be in thermal connection with the urine sample cell. The thermal sensor may detect the presence of urine in the urine sample cell by detecting a temperature that is in the range of body temperature, for example between about 90° F. and about 105° F. The thermal sensor may be in electrical connection with a controller which may actuate the fiber optic spectrometer to conduct a spectral analysis of the urine. A light emitting fiber and a light receiving fiber may connect the urine sample cell with the fiber optic spectrometer as disclosed elsewhere herein.

The urine sample cell may include a urine exit aperture which may be reversibly covered by a urine exit cover. Mechanisms disclosed herein may open and close the urine exit cover to contain or release either urine or flush water dispensed by a flush water dispenser to rinse the system between uses. The flush water dispenser may comprise a directional nozzle to efficiently direct the flow of flush water into the urine capture basin.

The controller may include machine-readable storage medium for storing historical urine analysis data and non-transient computer readable medium which may be programmed to analyze the spectral data. The non-transient computer readable medium may also compare a user's urine analyte levels with reference databases, with urine analyte levels of other users in a demographic group or geographic location, with disease markers that include urine analyte levels, and perform a historical analyses of a user's urine analyte levels assessed over time thus creating a longitudinal assessment of the user's urine metabolites.

### BRIEF DESCRIPTION OF THE DRAWINGS

FIG. 1A is an overhead view of a health monitoring toilet system depicting the internal arrangement of the various components according to an embodiment.

FIG. 1B is a side sectional view of a health monitoring toilet system depicting the internal arrangement of the various components according to an embodiment.

FIG. 2 is a diagram depicting the system for processing and storing results obtained from the health monitoring toilet system and the method for assessing, monitoring, and predicting the health status of the user, wherein the urinary component concentration calculation is used to identify disease markers, analyze trends, and evaluate the overall health status or disease state risk of the user.

FIG. 3 is a diagram depicting the system for processing and storing results obtained from the health monitoring toilet system and the method for assessing, monitoring, and predicting the health status of the user, wherein the user is identified using a urinary fingerprint analysis.

FIG. 4 is an aerial view of an embodiment of the health monitoring toilet system illustrating the urine capture basin in the toilet bowl and fiber optic connections to a spectrometer.

FIG. 5 is a cross-sectional view of the health monitoring toilet system of FIG. 4.

FIG. 6 is a schematic drawing of an embodiment of the urine sample cell within the disclosed health monitoring toilet system.

FIG. 7 is a schematic drawing of an embodiment of the urine sample cell within the disclosed health monitoring toilet system.

It will be appreciated that the drawings are illustrative and not limiting of the scope of the invention which is defined by the appended claims. The embodiments shown accomplish various aspects and objects of the invention. It is appreciated that it is not possible to clearly show each element and aspect of the invention in a single figure, and as such, multiple figures are presented to separately illustrate the various details of the invention in greater clarity. Similarly, not every embodiment need accomplish all advantages of the present invention.

### DETAILED DESCRIPTION OF THE INVENTION

### Definitions

User, as used herein, means an individual who has deposited urine, feces, or urine and feces in the disclosed health monitoring toilet system. The user may be animal or human.

Toilet, as used herein, means a device that may be used to collect urine, feces or urine and feces from a user. This may include a traditional water toilet. However, toilet, as used herein, may mean any device which may be used to collect urine and/or feces according to the present disclosure and which may be equipped to analyze urine and/or feces.

While this invention is susceptible of embodiment in many different forms, there are shown in the drawings, which will herein be described in detail, several specific embodiments with the understanding that the present disclosure is to be considered as an exemplification of the principals of the invention and is not intended to limit the invention to the illustrated embodiments.

Reference in the specification to “one embodiment” or “an embodiment” means that a particular feature, structure, or characteristic described regarding the embodiment is included in at least one embodiment, but is not a requirement that such feature, structure, or characteristic be present in any particular embodiment unless expressly set forth in the claims as being present. The appearances of the phrase “in one embodiment” in various places may not necessarily limit the inclusion of a particular element of the invention to a single embodiment, rather than element may be included in other or all embodiments discussed herein.

Furthermore, the described features, structures, or characteristics of embodiments of the invention may be combined in any suitable manner in one or more embodiments. In the following description, numerous specific details are provided to provide a thorough understanding of the invention. One skilled in the relevant art will recognize, however, that embodiments of the invention may be practiced without one or more of the specific details, or other methods, components, materials, and so forth. In other instances, well-known structures, materials, or operations are not shown or described in detail to avoid obscuring aspects of the invention.

It should be noted that, as used in this specification and the appended claims, singular forms such as “a,” “an,” and “the” may include the plural unless the context clearly dictates otherwise. Thus, for example, it is understood that a reference to “an engagement element” may include one or more of such engagement elements. In particular, with respect to the construction of claims, it is further understood that a reference to “an engagement element” reads on an infringing device that has more than one engagement element, since such infringing device has “an engagement element” plus additional engagement elements. Accordingly, the use of the singular article “a,” “an,” and “the” is considered open-ended to include more than a single element, unless expressly limited to a single element by such language as “only,” or “single.”

As used herein, the term “about” is used to provide flexibility to a numerical range endpoint by providing that a given value may be “a little above” or “a little below” the endpoint while still accomplishing the function associated with the range.

As used herein, a plurality of items, structural elements, compositional elements, and/or materials may be presented in a common list for convenience. However, these lists should be construed as though each member of the list is individually identified as a separate and unique member.

We disclose a system and method for assessing, monitoring, and predicting changes in physiological conditions, disease, and/or disease progression through ongoing and longitudinal analysis of various health-related parameters.

The data utilized for purposes of assessing, monitoring, and predicting changes in physiological conditions, disease, and/or disease progression may be obtained using various acquisition mechanism, for example, any suitable toilet or urinal, or other device designed to capture and analyze human waste may be used. Thus, although the application of such a system is shown in the context of a basic water toilet, it should be understood that other configurations are contemplated.

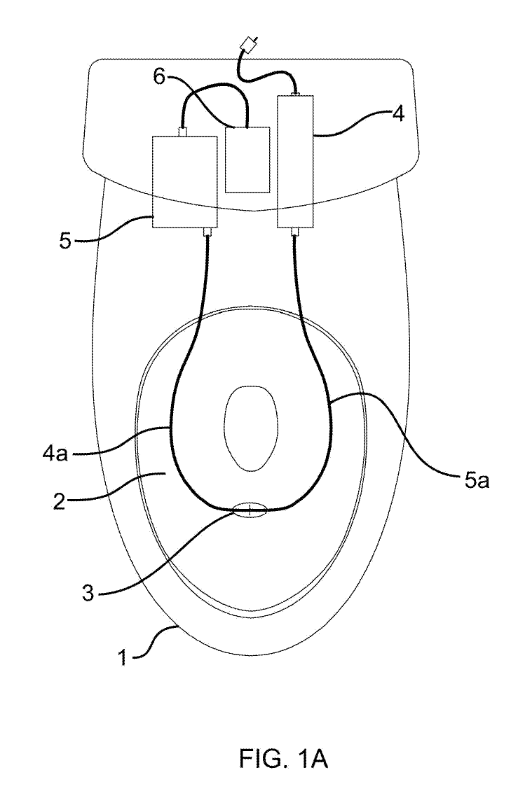

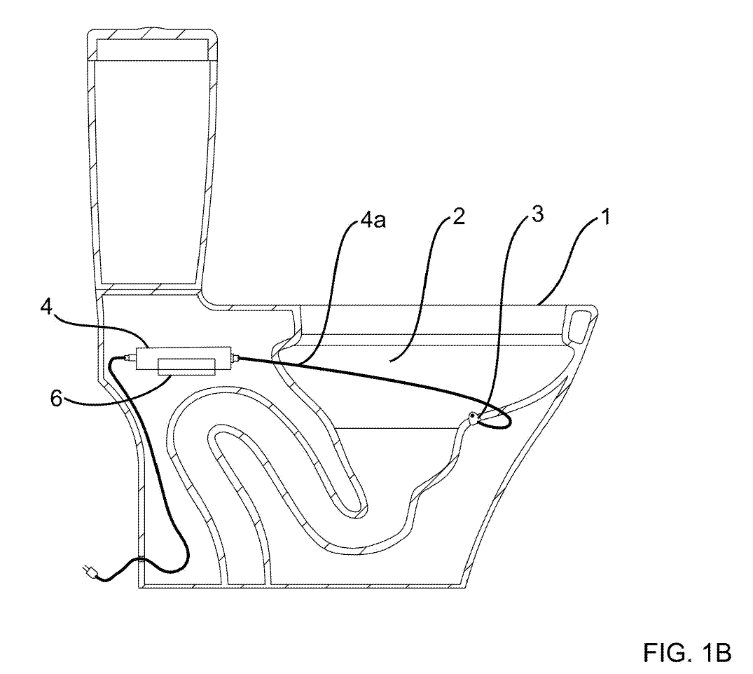

In one embodiment, the system comprises a near-infrared spectrometer integrated into a toilet, for example as shown in FIG. 1. A unique spectrum is obtained for each urine scan and chemometrically extrapolated to determine the concentration of a plurality of urine components. The concentration of these urine components, along with specific changes in the urinary spectra, form the basis for a centralized, continually updated reference database that may be used to assess, monitor, and predict health outcomes. New sample spectra and extrapolated concentrations may be compared against the reference database using statistical techniques to identify characteristics in keeping with diseased or non-diseased health states. Additionally, sample data may be compared on an ongoing basis against the user's own historical results to detect significant changes or trends in health status. By enabling ongoing longitudinal analysis of a broad range of health-related urinary parameters, the disclosed toilet-based health analysis system may assess, monitor, and predict the health of a user.

The present disclosure further relates to systems and methods for in vitro detection and evaluation of analytes in biological waste, including urine and/or feces, using one or more analytical tools incorporated into a toilet stool. For example, the toilet stool may employ a Raman spectroscopy system capable of irradiating a sample and producing a Raman spectrum comprising scattered electromagnetic radiation. Data collected may be processed by an integrated or remote processor to provide information about one or more analytes.

In one aspect, the present disclosure provides a health property monitoring system comprising a biological waste receptacle for collecting biological waste from a user; one or more analytical instruments connected to the biological waste receptacle and configured to analyze one or more properties of the biological waste collected by the biological waste receptacle; an electronic storage medium (hereinafter “machine-readable storage medium”) configured to store longitudinal data corresponding to the one or more properties of the biological waste, wherein the longitudinal data comprises a statistically significant plurality of data sets corresponding to individual biological waste samples collected over a period of time sufficient to establish a statistically significant baseline or trend of the one or more properties; and a computer processor (hereinafter, a “controller”) configured to determine a statistically significant attribute of the longitudinal data.

As described in detail herein, the health monitoring toilet system of the present disclosure provides a significant advance over presently available technology with respect to the information content of data received and processed by the system. In particular, the collection of longitudinal data over a period of time may provide a statistically significant plurality of data sets corresponding to individual biological waste samples collected over a period of time sufficient to establish a statistically significant baseline or trend of the one or more properties. A used herein, “statistically significant” means that a sufficient number of samples is obtained to achieve a confidence level that is statistically meaningful and representative of the condition or state of the user. In scientific terms, a statistically significant result is attained when a p-value is less than the significance level. The p-value is the probability of observing an effect given that the null hypothesis is true whereas the significance or alpha (a) level is the probability or rejecting the null hypothesis given that it is true. As a matter of good scientific practice, a significance level is chosen before data collection and is usually set to 0.05 (5%). Other significance levels (e.g., 0.01) may be used, depending on the field of study. Statistical significance is used as a measure of the probability of whether or not a data point or set of data points are consistent with a parent data set or fall outside parent data set norms. For example, urine urea values repeatedly acquired over multiple weeks should normally be distributed for a given individual. Based on the normal distribution, 95% of results should fall within two standard deviations of the mean urea value for the individual. For example, if 95% of an individual's urine urea concentrations fall between 1,500 and 2,000 mg/dL, a urine urea concentration of 1,990 mg/dL would not be considered a statistically significantly high value, at a significance level of 5%. However, a urine urea concentration of 2,010 mg/dL would be considered statistically significantly high because the likelihood that the measurement is due to random chance is less than 5%. The measurement is not reasonably explained by random variation. It will be apparent to those skilled in the relevant art that the bounds set for statistical significance will be set in accordance with various parameters; including, but not limited to: odds ratio, relative risk, variability of data, consequences of false positive and false negative results, or other relevant considerations. Thus, statistical significance is not limited to results having a p-value of less than 0.05. Accordingly, the importance of any given factor or set of factors will be determined individually and such determinations are known to those skilled in the relevant art as described, for example, by Munro, B., Statistical Methods for Health Care Research (Lippincott Williams & Wilkins, 2005). In accordance with such guidelines, those skilled in the art may select a p-value threshold to less than 0.05, for example, 0.04, 0.03, 0.02, or 0.01.

The term “longitudinal” means data that has been acquired over a period of time and represents a plurality of data points obtained at different times over such period of time, for example, days, weeks, months, or years. Longitudinal data may include, for example, data for a variety of trends and/or patterns, including, but not limited to, cyclic structures, periodicity, changes in levels over time as indicators of changing health condition, and/or variability over time as indicators of changing health condition.

In some embodiments of the health monitoring toilet system of the present disclosure, the longitudinal data corresponding to the one or more properties of the biological waste may further comprise a time component selected from one or more of a date, a time, and a frequency related to when the biological waste was collected. The time component of the data may indicate, for example, a date, a time of day, a season of the year, a year, etc. so that the data may be tracked chronologically and used to evaluate historical patterns of the user's health condition and predict future health conditions based on extrapolation of historical data.

It is further contemplated that in some embodiments, the health monitoring toilet system of the present disclosure may further comprise an input to receive a user identification corresponding to a source of the biological waste. The user identification may correspond to a single individual, and may comprise patient identifying information and non-identifying information, for example, demographic information. As described in further detail herein, the demographic information may be anonymized to protect the identity of the user.

In some aspect of the health monitoring toilet system of the present disclosure, the statistically significant attribute corresponds to data from a single individual. It is contemplated, for example, that a single individual may utilize the system of the present disclosure frequently, for example, multiple times per day, daily, multiple times per week, so as to obtain a set of data representing the health condition of the user over a sufficiently long period of time, with a sufficient number of data points, that it is possible to establish a statistically significant base line or trend that reflects the health condition of the user. The baseline may represent a healthy condition, from which the deviation represents a non-healthy condition. Alternatively, the baseline may represent a non-healthy condition from which the deviation represents a return to a healthy condition. Thus, in some embodiments, the longitudinal data comprises a statistically significant plurality of data sets corresponding to individual biological waste excretion events over a period of time sufficient to establish a statistically significant deviation from a baseline, a trend, or from a population or sub-population norm.

The health monitoring toilet system of the present disclosure may also be used to detect and analyze non-health conditions or disease states based on chemical variations or deviations prior to such variation or deviations presenting symptoms that are discernable to the user. Thus, in some embodiments, the statistically significant deviation constitutes a statistically significant pre-symptomatic deviation from the healthy condition. It is further contemplated that in some embodiments, the statistically significant deviation constitutes a statistically significant post-symptomatic deviation, or a deviation indicative of the future progression of a health or disease state.

In some embodiments, it is desirable that the health monitoring toilet system be configured to notify health care professionals or the user of changes in the health status that may be important to the health of the user. Thus, in some embodiments, the health monitoring toilet system may further comprise a diagnostic routine configured to send an electronic diagnosis of the statistically significant deviation to a designated recipient. In other embodiments, the controller may further comprise a notice routine configured to send an electronic notice of the statistically significant deviation to a designated recipient. Many disease states, for example, demonstrate improved response to treatments when initiated earlier in the disease process. Accordingly, an early warning system may be useful in developing more effective treatment regimens.

In accordance with the present disclosure, the controller may be configured to communicate with a plurality of biological waste receptacles as disclosed herein. In some embodiments, the individual units within the plurality of biological waste receptacles are electronically and communicatively connected, thereby enabling an individual user's data to be collected from the plurality of biological waste receptacles, (i.e., one at work, another at home, another in an airport, etc.) and pooled into a single data system so as to increase the number and frequency of relevant data points and thereby increase the power and accuracy of the data to establish a norm or trend away from the norm.

Accordingly, in some embodiments, each of the plurality of biological waste receptacles may comprise an input device to receive a user identification corresponding to an individual user who is the source of the biological waste. When a user deposits biological waste into a particular biological waste receptacle, the biological waste receptacle may be configured to identify the user and correlate the user with the data corresponding to the analysis of that user's biological waste. Similarly, some biological waste receptacles that are used by more than one user may be configured to identify more than one user. Accordingly, in other embodiments, the individual units in the plurality of biological waste receptacles may each comprise an input device to receive a user identification corresponding to an individual user who is the source of the biological waste, wherein the data comprises a plurality of user identifications corresponding to a plurality of users.

Where the health monitoring toilet system of the present disclosure is used to collect data from a plurality of users, it is possible then to track data corresponding to the plurality of users, for example, a group of individuals in a common home, common work environment, common hospital, common zip code, common city, common geographical region, etc., which may enable the health monitoring toilet system to identify norms and trends in such population or sub-population, or as compared to other populations. Accordingly, in some embodiments, the present disclosure provides a health monitoring toilet system wherein the plurality of users comprises a sufficient number of users to establish a statistically significant population or sub-population norm. In other embodiments, the data is derived from a sufficient number of users to determine a statistically significant deviation from the population or sub-population norm. For example, in some embodiments, the health monitoring toilet system of the present disclosure may track data from a discrete sub-population group comprising a single home, a medical practice group, hospital, school, prison, or business group. Sub-populations may also include, for example, sub-populations defined according to age, blood glucose, body-mass index, current and past medications, diagnoses of a particular disease, dietary patterns, elevation, gender, general geographic location, height, independent laboratory results, medical diagnostic test results, medical history, race, temperature, wearable device results, weight, or any other relevant factor related to health or disease states.

In some embodiments, the controller further comprises a notice routine configured to send an electronic notice of the statistically significant deviation to designated recipient. In other embodiments, the data is derived from a sufficient number of users to establish a statistically significant norm of a discrete sub-population group.

FIG. 1A and FIG. 1B depict a health monitoring toilet system which may be used to quantify the concentrations of a multiplicity of urinary components in an automatable, reagent-free manner which is readily amenable to domestic or other on-site environments, thereby allowing for acquisition of the continuous measurements necessary to assess, monitor, and predict the health status of the user.

A toilet body 1 has a toilet bowl 2, a urine sampling device 3, a light source part 4, a light measuring part 5, and a computing and transmitting part 6. In the depicted embodiment, the urine sampling device 3 which is integrated into toilet bowl 2 is provided with a urine sampling cell, such that urine flowing across the toilet bowl 2 passes over the urine sampling device 3 and through the urine sampling cell. The urine sampling cell contains a thermistor for detecting when urine has been introduced into the urine sampling cell by means of a temperature change resulting from the presence of urine. In some embodiments, the thermistor may detect a specific range of temperatures consistent with a normal body temperature of a user, for example, ranging from about 90° F. to about 106° F., or alternatively from about 97° F. to about 100° F., or alternatively from about 97.7° F. to about 99.5° F.

A light source part 4 is provided for irradiating the urine sample cell with a measuring beam, while a light measuring part 5 is provided for receiving and detecting the measuring beam transmitted through the urine sampling cell. The measuring beam is conducted from the light source part 4 to the urine sample cell through a light emitting fiber 4a and is conducted from the urine sample cell to the light measuring part 5 through a light receiving fiber 5a. The light source part 4 and the light measuring part 5 serve both as means for measuring absorbances of a urine sample in the urine sampling cell of the urine sampling device 3 and as a sensor for detecting soiling of the urine sample cell by measuring changes in the absorbance of the cell itself in order to determine the degree of soiling of the urine sample cell.

The light source part 4 comprises a lamp source emitting a light of a continuous range of wavelengths, a light-emitting diode array emitting light of a continuous range of wavelengths, a laser unit having a variable oscillation wavelength, or a laser diode array emitting laser beams for measuring wavelengths. The light measuring part 5 is provided with a spectrometer component or interferometer component and photodetector component comprised of a photodiode, an array type photoreceptor of CCD, a photoreceptor array, or a single photoreceptor as a detector. Light intensity or quantity measurement sensitivity depends on optical path lengths and wavelengths. The urine sample cell is not restricted to a single optical path length, but may be provided with continuously or step-wisely differing optical path lengths chosen in a manner that optimizes the signal-to-noise ratio for a given wavelength or set of wavelengths. Additionally, measuring time may be used to improve signal-to-noise ratio for a given wavelength, set of wavelengths, or the spectra as a whole and may be chosen from the time range of 10 to 1,800,000 ms. Following emission from the light emitting fiber 4a, the measuring beam is transmitted through the urine sample cell and is received by the light receiving fiber 5a, so that the measuring beam transmitted through the cell is spectroscopically analyzed by the spectrometer component of light measuring part 5 and thereafter guided to the photoreceptor component of light measuring part 5.

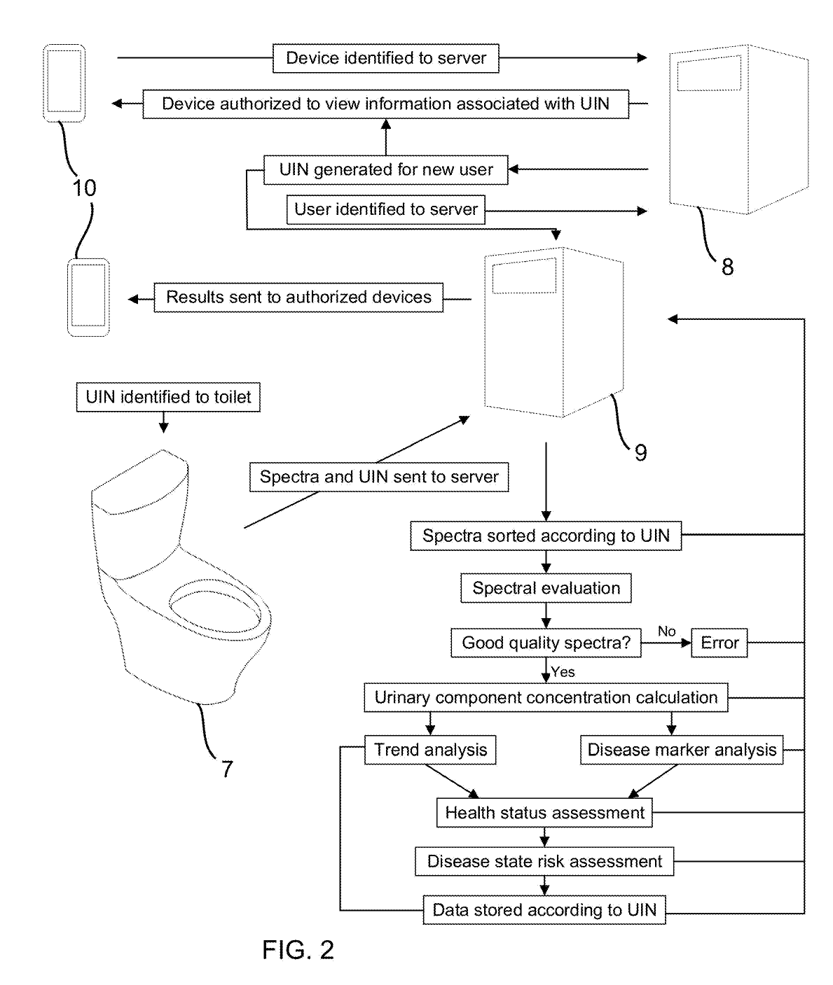

FIG. 2 and FIG. 3 illustrate the system by which absorbance data is transmitted, stored, and interpreted, and thereby providing continuous health assessment, monitoring, and prediction for the user. The system comprises the elements of a toilet body 7, a remote identifying information server 8, a remote data storage and analysis server 9, and an electronic computing device 10 owned and maintained either by the user or a party authorized by the user to receive the user's health-related information.

Individually identifiable information, for example, name, address, billing information, or date of birth, is stored on the remote identifying information server 8 for each unique user and each unique user is assigned a unique identification number (hereinafter “UIN”). Other user-related data may also be associated with the UIN, for example, gender, race, nationality, socioeconomic status, residential zip code, veteran status, disease biomarker status, etc., which data may be useful in interpretation of population or sub-population studies. This UIN is communicated to the remote data storage and analysis server 9. Additionally, an electronic computing device 10 or multiple devices may be authorized by the user to receive the user's health-related information. This electronic computing device 10 receives a digital authorizing certificate from the remote identifying information server 8 allowing the electronic computing device 10 to retrieve health-related information associated with the user.

Health state assessment, monitoring and prediction is initiated when a user is identified to the toilet body 7. This identification may occur using a variety of means including direct entry of the UIN via a built-in, wired, or wireless keypad; wireless pairing with an authorized electronic computing device 10; recognition of implanted, worn, or carried radio frequency identification (RFID); or fingerprint, retinal scan, or other biometric identification. The subsequent urine sample spectra obtained using the toilet body 7 are then coupled with the supplied UIN and wirelessly transmitted to the remote data storage and analysis server 9.

Following receipt of new data from toilet body 7, the remote data analysis server 9 sorts the spectral data in accordance with the accompanying UIN. Spectra are then evaluated to determine whether or not they meet basic quality parameters. Spectra of sufficient quality undergo algorithmic processing because of the absorbances measured in the toilet body 7 to obtain urinary component concentrations. Spectra of insufficient quality are designated as erroneous and recorded as such. In order to measure a multiplicity of urinary components, measuring wavelengths are selected which are best correlated with urinary component concentrations as measured by a preexisting assay. Wavelengths or wavelength regions having absolute values of correlation coefficients of at least 0.4 to a chosen urinary component are regarded as measuring wavelength regions and are selected from 100 nm to 4,000 nm wavelength range. Additionally, wavelengths or wavelength regions having absolute values of correlation coefficients of at least 0.1 to the presence, absence, or severity of the disease, disease state, health risk factor, or other health state are regarded as measuring wavelength regions and are selected from 100 nm to 4,000 nm wavelength range.

Urinary component concentrations are then evaluated by the remote data analysis server 9 and classified as “normal” or “abnormal.” The remote data analysis server 9 compares the most recently obtained data associated with a UIN with historical data associated with same UIN to establish trends over time. Urinary components for an individual which have an overall regression slope of less than 0.2 measurement units per time unit, as measured across multiple appropriate time intervals, are defined as “normal” for that individual. Urinary components, which have an overall regression slope of 0.2 measurement units per time unit or greater, as measured across multiple appropriate time intervals, are defined as “abnormal” for that user. The remote data analysis server 9 also assesses urinary component results to determine if results are direct markers of disease, disease state, health risk factor, or other health state as determined by predefined minimum or maximum healthy values for a healthy individual.

The aggregate of trend analysis and disease marker analysis is then employed by the remote analysis server 9 to determine the current health status of the user. Changes in trend or disease state markers or in the health status of the user are then used to evaluate the risk that the user will develop a particular disease state within a given time frame. These changes and their significance may be identified using a variety of statistical techniques including, but not limited to, partial least squares or principal component regression, although a variety of other techniques may be employed; including, but not limited to: artificial neural networks, multiple linear regression, multivariate curve resolution, support vector machine classification or regression or cluster analysis. Alternatively, machine learning or statistical techniques familiar to those skilled in the art may be employed to identify other predictive aspects derived from continuous monitoring of urine samples. Non-component-specific changes in the urinary spectra may also be evaluated as predictors of changes in components of bodily fluids other than urine or general changes in health status. These predictors have absolute correlation coefficient values between changes in urinary spectra and changes in bodily fluid components or health conditions of at least 0.2. This analysis may be accomplished by the remote data analysis server 9 concurrent with the evaluation of spectral quality.

Following data analysis, the remote data analysis server 9 stores spectral quality and analysis results, urinary component concentrations, trend and disease marker results, health assessment findings, and disease risk results in accordance with their associated UIN. These results may then be accessed by an electronic computing device 10 authorized to view data associated with the appropriate UIN. A rules engine for determining which parameters dictate transmission of an alert to an authorized electronic computing device 10 may be defined on the authorized electronic computing device 10.

In some embodiments of the present disclosure, measurements collected from the sampling site are communicated wirelessly to a remote server for processing and storage. Each user is assigned a unique dentification number (UIN) that pairs spectral data from a given urine or fecal sample with the individual who produced the sample. The system identifies an individual by one or several alternative means; including, but not limited to: direct entry of the UIN via a built-in, wired, or wireless keypad; wireless paring with a user-owned cellular device; recognition of implanted, worn, or carried radio frequency identification; or fingerprint, retinal scan, or other biometric identification.

Once the user and their associated UIN have been identified, the UIN is used to link spectra, predicted urinary or fecal component concentrations and other non-identifying health information related to a specific user. Non-identifying health information may include, but is not limited to: age, blood glucose, blood pressure, body-mass index, current and past medications, diagnoses, dietary patterns, gender general geographic location, height, independent laboratory results, medical diagnostic test results, medical history, race, temperature, wearable device results, or weight. This information may be electronically communicated to the server directly by the user or the user's healthcare provider. Alternatively, the server may be linked to the user's patient file, electronic health record, or other medical database, allowing for online communication of health data. Information may also be added from an independent device used to track the previously described elements or to facilitate documentation of other health-related parameters.

A separate server is used to store individually identifiable information, including, but not limited to, name, address, or billing information in coordination with the user's UIN. This server issues digital certificates of authorization to the computer, smart device or other electronic devices of the user or another individual or group authorized by the user. These certificates authorize the electronic device to retrieve personal health information associated with the authorized UIN from the previously mentioned remote server. As a result, breach of a single server will not provide both individually identifiable and health information.

Once spectral data has been assigned to the proper user, values at specific points are algorithmically extrapolated to generate the predicted concentrations of urinary or fecal components in a sample or to identify the presence, absence, or severity of a disease, disease state, health risk factor, or other health state for the sampled individual. To avoid faulty data, scans may be discarded if values at specified points lie outside predetermined minimums and maximums. Results are stored as previously mentioned and all results are preferably plotted as a time series. Since all possible algorithmic extrapolations may not be identified prior to sampling, stored spectra may also be retroactively reprocessed using algorithms developed subsequent to sample acquisition to determine historic concentrations of urinary or fecal components in one or more samples or to identify the historic presence, absence, or severity of a disease, disease state, health risk factor, or other health state for the sampled individual. In addition to the other health-related information elucidated by the disclosure, the ability to retroactively assess samples for previously unidentified health changes provides a heretofore impossible means for following the course of disease and health.

Data assignation, extrapolation, and sequencing allow health parameters present in urine to be tracked and monitored in real-time. This offers numerous advantages over current methodology. First, daily or multi-daily tracking of urinary or fecal components may be used to identify a user's true normal range over time. Currently, test results from a single point in time are used to determine an individual's relative health; however, Knuiman et al. (1986) reported in Human Nutrition Clinical Nutrition, 40, 343-348 that it required 4-14 days of continuous 24-hour sampling to estimate urinary components to within 20% of habitual excretion. Knuiman et al. (1988) reported similar results in Clinical Chemistry, 34, 135-138, with the added observation that it required 11-26 days, depending on the specific urinary component, or sequential overnight urine sampling to accurately estimate urinary components to within 20% of habitual excretion. Overnight urine testing is far more similar to the routine sampling protocols employed by the medical profession than continuous 24-hour sampling; therefore, since the within-person variability reported in this study ranged from 33-52% for overnight testing, the current inability to acquire numerous sequential samples means that the single-point test results used by healthcare professionals to monitor and treat an individual's health are poor estimates of that individual's typical urinary component concentration. This highlights the utility of the disclosed innovation, which, by eliminating the difficulties of conventional urine and fecal testing, makes accurate assessment of an individual's urinary or fecal component concentrations routinely achievable through ongoing monitoring.

Second, daily or multi-daily tracking of urinary components may be used to identify “normal” and “abnormal” trends in urinary or fecal component concentration. Given the human body's predilection to maintain homeostasis, a regression line plotted across the sequential urinary concentrations of various components has an effective slope of zero, given an appropriate time window. There may be a sinusoidal component to the production and/or excretion of certain urinary or fecal components which may follow circadian, diurnal, nocturnal, monthly, or other biological rhythms; however, the overall slope across multiple cycles for these components remains approximately zero under stable health conditions.

In the present innovation, urinary or fecal components for an individual which have an overall regression slope of less than 0.2 measurement units per time unit, as measured across multiple appropriate time intervals, are preferentially defined as “normal” for that individual. This may or may not be substantively different than the normal for the population as a whole. In contrast, an individual's urinary or fecal components which have an overall regression slope of 0.2 or greater measurement units per time unit, as measured across multiple time intervals, are preferentially defined as “abnormal” for that individual. In this way, the disclosed innovation may be used to identify consistent changes in health, regardless of the presence or absence of symptoms. Whether positive or negative, these changes in excretion represent changes in the fundamental health processes of the user.

Third, disease markers change in advance of observable symptoms; therefore, daily or multi-daily tracking of urinary or fecal components enables pre-symptomatic diagnosis and treatment. For example, kidney stones form subsequent to well-defined changes in urinary components. The solubility of calcium oxalate—the key precipitate in 80% of nephrolithiasis cases—in water is about 0.44 mg/dL; however, this is mitigated by the presence of citrate, which complexes with free calcium ions and inhibits the formation of calcium oxalate crystals. Kidney stones frequently form when the urinary concentration of oxalic acid is consistently above 0.44 g/dL and citric acid excretion is below 325 mg/24 h. Since the free crystallization of renal calculi takes time, continuous monitoring of these urinary components may be used to identify patients at significant risk for kidney stones before the condition becomes symptomatic. Dietary or medical interventions may then be implemented to reverse the crystallization process, allowing the individual to return to a healthy state and circumvent the discomfort of passing a kidney stone. While these predisposing changes in urinary component concentrations have been known for decades, current medical testing is unable to supply the real-time monitoring needed to pre-symptomatically identify and treat nephrolithiasis. This example is representative of many other disease states in which symptoms are preceded by urinary or fecal component concentrations; however, without a system for continuous monitoring of these components, these changes are typically only used in confirmatory testing after symptoms have developed.

Fourth, daily or multi-daily tracking of urinary or fecal components may be used to identify new links between changes in urinary component concentration and the development, progression, or exacerbation of a disease state. For example, Loureiro et al. (2014) reported in the Journal of Allergy and Clinical Immunology, 133, 261-263 that principal component analysis of urine component concentrations revealed that threonine, alanine, carnitine, trimethylamine-N-oxide, and acetylcarnitine concentrations increased and acetate, citrate, malonate, phenylacetylglutamine dimethylglycine, and hippurate concentrations decreased during asthma exacerbations. Loureiro et al. concluded from their findings that changes in these or other urinary components could be used to predict the onset of an asthma exacerbation. Similarly, Liang et al. (2009) reported in Guan Pu Xuan Yu gang Pu Fen Xi, 29, 1772-1776 that Bayes stepwise integration of NIR spectra enabled them to correctly identify chronic enteritis in alpine musk deer with 100% accuracy and identify healthy specimens with 93.3% accuracy. These examples are representative of many other disease states which effect metabolic changes that can be monitored in the urine or feces.

In addition to finding new correlations between disease states and alterations in urinary or fecal component concentrations, continuous monitoring of urinary or fecal spectra may be used to identify wavelengths or groups of wavelengths that vary consistently in accordance with changes in an individual's health condition or the molecular makeup of other body systems of fluids. For example, Purnomoadi et al. (2000) reported in Near-Infrared Spectroscopy: Proceedings of the 9^{th} International Conference, 729-733 a correlation coefficient of 0.96 between a urinary absorbance peak located at 2134 nm and the blood urea nitrogen of cows. This wavelength remained highly predictive when cows' blood urea nitrogen increased in response to stress. Thus, the continuous monitoring provided by the present invention may be used to identify changes in health either directly through urinary component quantification or indirectly through changes in the urinary spectra.

In one embodiment, correlations between changes in urinary or fecal spectra and changes in bodily fluid components or health conditions of at least 0.2, preferably 0.6, are identified using partial least squares or principal component regression, although a variety of other techniques may be employed; including, but not limited to: artificial neural networks, multiple linear regression, multivariate curve resolution, support vector machine classification or regression or cluster analysis. Alternatively, machine learning or other statistical techniques familiar to those skilled in the art may be employed to identify other predictive aspects derived from continuous monitoring of urine or fecal samples.

Lastly, changes in urinary or fecal spectra may be used to monitor drug usage and metabolism. The vast majority of drugs and their metabolites are excreted to some extent in urine and virtually all drugs and the metabolites not excreted in urine are excreted in feces. Thus, by continuously monitoring urine and/or feces, it is possible to determine drug usage and metabolism. Currently, the cost of monitoring drug usage and metabolism is prohibitively time-consuming and expensive. Since drug usage and metabolism are crucial to therapeutic decision-making, the proposed invention offers an unprecedented way to rapidly determine the usage and monitoring for illicit drug usage, thereby both improving compliance with prescribed drugs and constraining misuse of drugs.

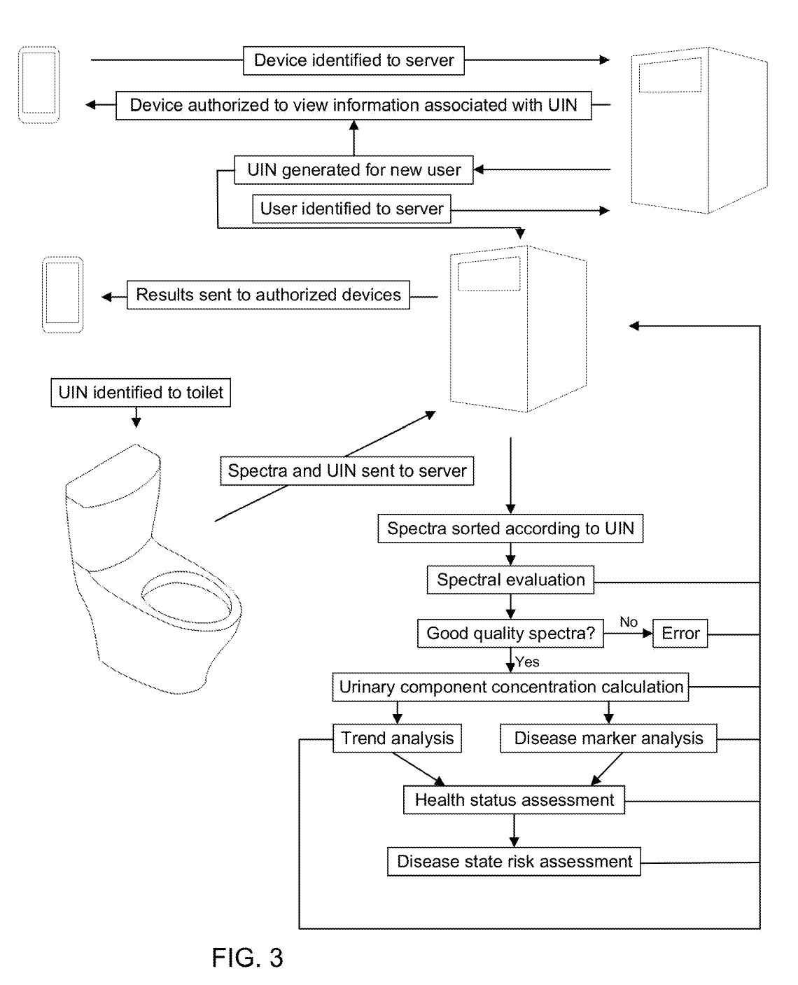

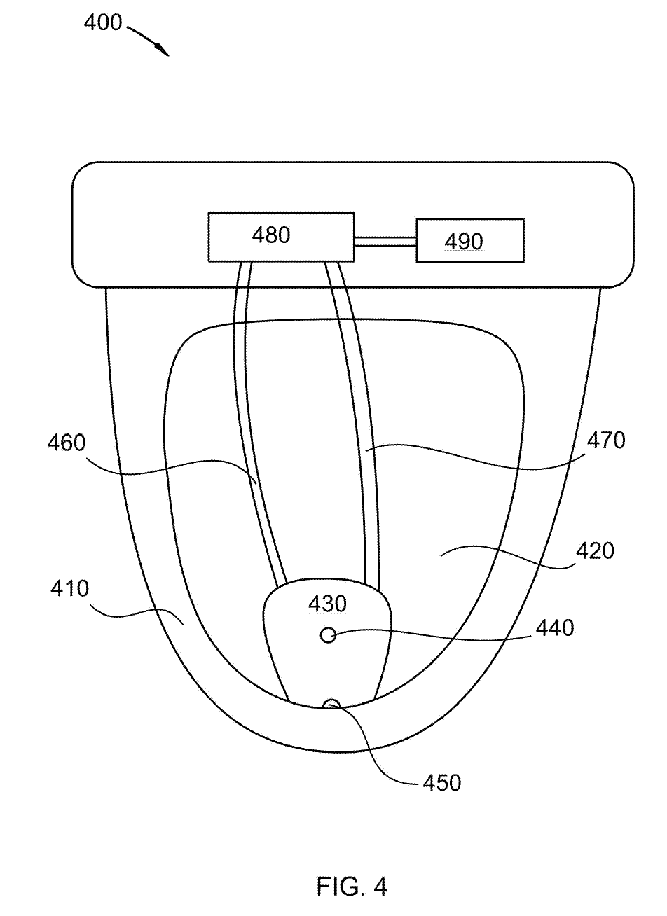

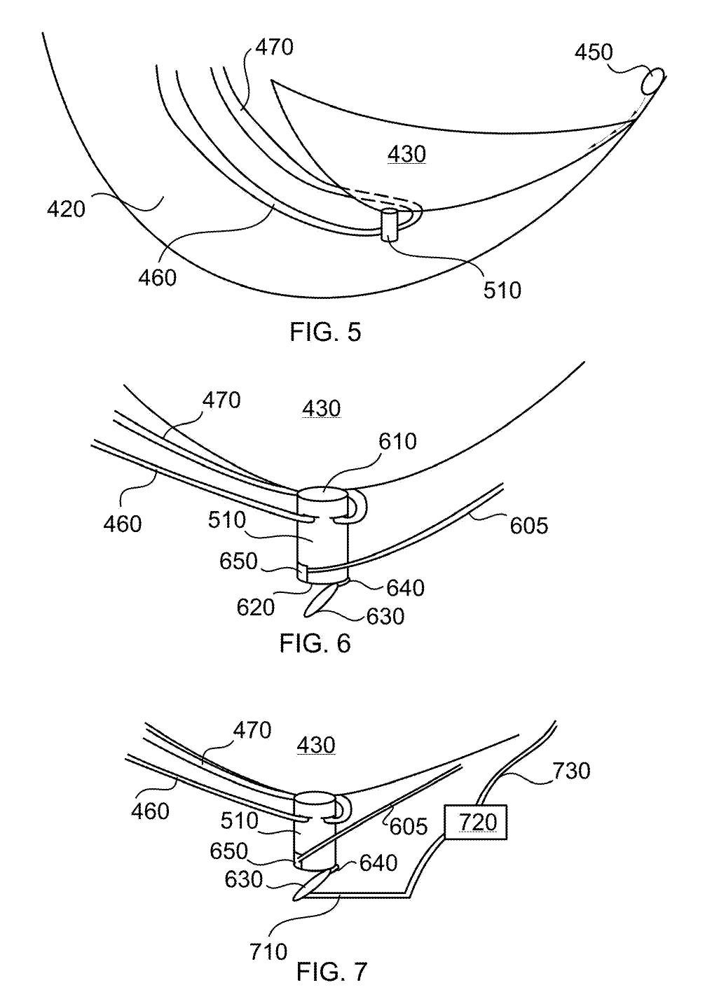

FIG. 4 illustrates an aerial view of an embodiment of the disclosed health monitoring toilet system, toilet system 400. Similar to traditional water toilets, toilet system 400 includes toilet rim 410 and toilet bowl 420. Urine capture basin 430 is shown within toilet bowl 420 and includes a urine entry aperture 440 through which urine may flow into a urine sample cell (shown in FIGS. 5 and 6). The front edge (toward the lower end of the drawing) of the upper rim of urine capture basin 430 is in contact with toilet bowl 420. After each use, flush water dispenser 450 may dispense water into urine capture basin 430 which may then flow through urine entry aperture 440 to cleanse the system between uses. The flush water dispenser may comprise a directional nozzle to more efficiently direct the flow of water into the urine capture basin. Light emitting fiber optic cable 460 transmits light from fiber optic spectrometer 480 and light receiving fiber optic cable 470 conducts light passing through the urine sample cell and transmits the light back to the fiber optic spectrometer 480 where the wavelength may be measured. Controller 490 is in electrical connection with fiber optic spectrometer 480. Controller 490 may include machine-readable storage medium for storing spectral data collected by fiber optic spectrometer 480 and non-transitory computer readable medium for analyzing and transmitting this data. Controller 490 may include a communication port capable of transmitting data from the controller to an external database.

FIG. 5 illustrates a side view of toilet system 400 first shown in FIG. 4. Urine capture basin 430 is again shown within toilet bowl 420. Urine capture basin 430 comprises an upper rim, the upper rim having a forward edge. The forward edge is in connection with the front of toilet bowl 420 (right side of the drawing). Flush water dispenser 450 is disposed on an inner wall of toilet bowl 420 and dispenses flush water into urine capture basin 430 as shown by the arrows. The flush water may then enter urine sample cell 510 to cleanse the system in between uses. During use, a user may urinate normally into toilet bowl 420 and urine capture basin 430 may capture some or all the urine. Gravity may direct the urine downward into urine sample cell 510 which includes a urine entrance aperture at the top (shown in more detail in FIG. 6). During urine analysis, light emitting fiber optic cable 460 transmits light from fiber optic spectrometer 480 shown in FIG. 4 (omitted in FIG. 5 for clarity) and light receiving fiber optic cable 470 conducts light passing through the urine sample cell and transmits the light back to the fiber optic spectrometer 480 where the wavelength may be measured.

FIG. 6 provides a close-up and more detailed illustration of an embodiment of urine sample cell 510. Urine sample cell 510 includes urine entry aperture 610 through which urine may flow from urine capture basin 430 and which is located at the upper end of urine sample cell 510. Light emitting fiber optic cable 460 transmits light from fiber optic spectrometer 480 shown in FIG. 4. Light passes through urine sample cell 510 and the urine within, then exits urine sample cell 510. Light receiving fiber optic cable 470 conducts light passing through the urine sample cell and transmits the light back to the fiber optic spectrometer 480 where the wavelength may be measured. The embodiment of FIG. 6 further includes thermistor 650 which is in electrical connection with controller 490 (shown in FIG. 4) through cable 605. Thermistor 650 measures the temperature of the contents of urine sample cell 510. When thermistor 650 detects a temperature that approaches body temperature, for example, between about 90° F. and about 105° F., and sends the detected signal to controller 490, the signal may be interpreted to mean that urine is present in urine sample cell 510. Controller 490 may then actuate fiber optic spectrometer 480 to initiate a spectral measurement.

Urine sample cell 510 of FIG. 6 further includes urine exit cover 630 which is connected to urine sample cell 510 through hinge 640 and reversibly covers a urine exit aperture 620 located at the lower end of urine sample cell 510. Urine exit cover 630 may open and close by rotating on hinge 640. When urine exit cover 630 is closed, urine is confined to urine sample cell 510 were it may be analyzed as described herein. When urine exit cover 630 is open, urine may flow out of urine sample cell 510, through the urine exit aperture 620, and into toilet bowl 420 for disposal. In the embodiment of FIG. 6, the weight of the liquid in urine sample cell 510 applies pressure to urine exit cover 630 causing it to open. The tension in the spring in hinge 640 may be adjusted to allow urine exit cover 630 to open when a defined volume or weight of fluid is present in urine sample cell 510. Consequently, as urine flows into urine capture cell 510, a spectral reading may be taken. As urine continues to flow into urine sample cell 510, the pressure on urine exit cover 630 causes it to open and the urine exits out the lower end of urine sample cell 510 through the urine exit aperture 620. Flush water dispenser 450 shown in FIG. 4 may dispense flush water which may enter urine sample cell 510 in amounts that cause urine exit cover 630 to open. The flush water then exits urine sample cell 510 along with any residual urine clinging to urine capture cell 510.

FIG. 7 illustrates another embodiment of urine capture cell 510. The embodiment of FIG. 7 is similar to that of FIG. 6 with urine exit cover 630 connected to urine sample cell 510 through hinge 640. However, the embodiment of FIG. 7 includes arm 710 which connects urine exit cover 630 to motor 720. When actuated, motor 710 may move arm 710 laterally to open and close urine exit cover 630. Cable 730 is an electrical connection between motor 710 and controller 490. Similar to the embodiment of FIG. 6, when thermistor 650 senses that the contents of urine sample cell 510 approaches body temperature, for example, between about 90° F. and about 105° F., the signal may be transmitted to controller 490 through cable 605. Controller 490 may receive the signal and actuate fiber optic spectrometer 480 which may take a spectral reading of the urine in urine sample cell 510. When the reading is complete, controller 490 may signal motor 720 to actuate arm 710 to move laterally and cause urine exit cover 630 to open. Urine may flow out of urine sample cell 510 and into toilet bowl 420. Flush water dispenser 450, which may comprise a directional nozzle, shown in FIG. 4 may dispense flush water which may pass through urine sample cell 510 rinsing away residual urine. Thermistor 650 may detect the lower temperature of the flush water and may send a signal through cable 605 to controller 480. Controller 480 may then send a signal to motor 720 which may actuate and cause arm 710 to move laterally. This lateral movement may cause urine exit cover 630 to close in preparation for the next sample reading.

It will be appreciated that thermistor 650 may comprise of other temperature detectors known in the art. For example, the temperature sensor may include, but is not limited to, a silicon band gap temperature sensor, a negative temperature coefficient thermistor, a resistance temperature detector, or a semiconductor based sensor.

In addition, the embodiments shown in FIGS. 4-7 may also include a user identification input device which may receive a user identification corresponding to an individual user who is the source of the biological waste as described elsewhere herein. In some embodiments, the user identification input device may include one or more of a smartcard scanner, radio frequency identification reader, a near field communication transaction device, or a numerical input pad. In some embodiments, the user identification input device is a biometric sensor. In some embodiments, the biometric sensor may be a fingerprint recognition sensor, a retinal scanner, or an iris scanner.

As discussed with reference to other embodiments, the controller 490 in embodiment of FIGS. 4-7 may include machine-readable storage medium which may store analyses collected over time from a user and also from a plurality of users, each being stored in a separate file assigned to each user. Controller 490 may further include non-transitory computer readable medium which may be programmed to conduct statistical analyses as described herein and assess trends in urine components. The non-transitory computer readable medium may compare the analysis of a user's urine to a reference database comprising ranges of normal urine metabolite values. The ranges of normal urine metabolite values may be derived from historical readings from the user when the user was known to be in a state of good health or readings from other users stored by the machine-readable storage medium. The non-transitory computer readable medium may further compare the values of the user's urine metabolites with a database of disease indicators which include aberrant values of urine metabolites.

As mentioned above, controller 490 may include a communication port capable of transmitting data from the controller to an external database. The communication port may receive data from an external database which comprises data collected from urine analyses form other users. In some embodiments, the controller may receive analyses collected from other health monitoring toilet systems which may be connected through a network.

Embodiments disclosed herein may include a combination of one or more analytical tools with their associated reagents an any variants or new and/or alternative analytical techniques designed for use with those tools as recognized by those skilled in the art of laboratory analysis, including, but not limited to: Raman spectrometer, nuclear magnetic resonance (NMR) spectrometer, near infrared (NIR) spectrometer, infrared spectrometer, ultraviolet spectrometer, visible light spectrometer, gas chromatograph (GC), liquid chromatograph (CL), high performance liquid chromatograph (HPLC), mass spectrometer (MS), microscope, photographic camera, ion fuel-cell devices, ion-selective electrode, weight scale, Geiger counter, thermometer, pH gauge, flowmeter, colorimeter, enzyme electrode, enzyme-linked immunosorbent assay (ELISA), color sensor, test strips, oxidation-reduction reagents, precipitants, magnetometer, photometer, microbial growth media, refractometer, antibodies, and other reagents, Sampling for these tools which are preferentially positioned within the toilet, may occur at one or more sites in or on the toilet bowl and/or piston chamber.

In one embodiment, spectroscopic components may produce radiation and provide spectroscopic measurements of a urinary and/or fecal sample. For example, an 805 nm, focusable 800 mW laser may be directed to a sample through a 50/50 beam splitter and a microscope objective lens. This light is then passed through a notch filter, 50 μm slit, and plano/convex lens before it is focused onto a holographic diffraction grating to produce a spectrum. The resulting spectrum may be directed to a charge coupled device (CCD), generating a spectral image which may then be translated into a Raman signature using analytical software.

In one aspect of the disclosed health monitoring toilet system, test data may be combined with data uploaded by other users to examiner acute population ranges and a user's relative state within the actual population range. Test data may also be evaluated longitudinally to evaluate a user's relative state within population trends. In addition to unitary variable analysis, data may be examined for interactive (multivariate), exponential, logarithmic, and other effects. Combined data may be continuously evaluated for predictive or excludability potential.

In one aspect of the health monitoring toilet system, applications that collect non-diagnostic data that may be relevant to health may be integrated into the system's data.

In another aspect of the health monitoring toilet system, non-test data may be folded into the models both for predictive relevance and sometimes as the key measurable.

In one aspect of the health monitoring toilet system, users may be able to set personal preferences for a variety of features; including, but not limited to communications and alerts, test sensitivity and/or potential out-of-range conditions, PINs, information sharing, and/or specific health aspects they would like targeted for evaluations. Users may also be able to enter personal information into the system; including, but not limited to: name(s) of healthcare provider(s), health information, and insurance information.

In one aspect of the health monitoring toilet system, out-of-range conditions, low-probability changes to baseline metrics, trend changes, or other predictive results may generate an alert. The alert may be conveyed to a user based upon their preferences and may also be conveyed to others along with appropriate information based upon user preferences.

In one aspect of the health monitoring toilet system, health practitioners may have the ability to register with a system and become connected to their patient's health information, provided the patient authorizes such a disclosure. Practitioners may add their diagnoses and prescribe treatment to the system and see the impacts to patient health outcomes in real-time. These diagnoses and prescriptions may be added to an overall master database to assist in uncovering new trends and correlations.

While specific embodiments have been described above, it is to be understood that the disclosure provided is not limited to the precise configuration, steps, and components disclosed. Various modifications, changes, and variations apparent to those of skill in the art may be made in the arrangement, operation, and details of the methods and systems disclosed, with the aid of the present disclosure.

Without further elaboration, it is believed that one skilled in the art can use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art that changes may be made to the details of the above-described embodiments without departing from the underlying principles of the disclosure herein.

We claim:

1. A health monitoring toilet system comprising:
a. a toilet bowl, the toilet bowl comprising:
i. an inner wall; and
ii. at least one flush water dispenser, the at least one flush water dispenser disposed on the inner wall of the toilet bowl;
b. a urine capture basin, the urine capture basin comprising:
i. a concave shape;
ii. an upper rim, the upper rim comprising a forward edge, wherein the urine capture basin is disposed within the toilet bowl and below the at least one flush water dispenser, and wherein the forward edge is in contact with the inner wall of the toilet bowl;
c. a urine sample cell, the urine sample cell comprising:
i. an upper end and a lower end;
ii. a first side and a second side, wherein the first side is opposite the second side;
iii. a urine entry aperture, the urine entry aperture disposed on the upper end of the urine sample cell;
iv. a urine exit aperture, the urine exit aperture disposed on the lower end of the urine sample cell;
d. a fiber optic spectrometer, the fiber optic spectrometer comprising:
i. a light emitting fiber optic cable, wherein the light emitting fiber optic cable is connected to the first side of the urine sample cell; and
ii. a light receiving fiber optic cable, wherein the light receiving fiber optic cable is connected to the second side of the urine sample cell;
e. a controller, the controller comprising:
i. a non-transitory computer readable medium; and
ii. a machine-readable storage medium, wherein the controller is in electrical connection with the spectrometer.

2. The health monitoring toilet system of claim 1, further comprising a temperature sensor, wherein the temperature sensor is in thermal connection with a space within the urine sample cell.

3. The health monitoring toilet system of claim 2, wherein the temperature sensor consists of one of the following: a silicon band gap temperature sensor, a negative temperature coefficient thermistor, a resistance temperature detector, and a semiconductor based sensor.

4. The health monitoring toilet system of claim 2, wherein the temperature sensor is in electrical connection with the controller.

5. The health monitoring toilet system of claim 4, wherein the non-transient computer readable medium actuates the spectrometer upon receipt of a temperature sensor reading of between about 90° F. and about 105° F.

6. The health monitoring toilet system of claim 1, wherein the urine exit aperture is covered by a urine exit cover, wherein a hinge connects the urine exit cover to the urine sample cell.

7. The health monitoring toilet system of claim 6, wherein the hinge comprises a spring.

8. The health monitoring toilet system of claim 6, further comprising:
a. a motor; and
b. an arm, wherein the arm comprises:
i. a first end; and
ii. a second end; wherein the first end of the arm is connected to the urine exit cover, and wherein the second end of the arm is connected to the motor, and wherein the motor, when actuated, moves the arm causing the urine exit cover to open and close.

9. The health monitoring toilet system of claim 1, further comprising a user identification input device.

10. The health monitoring toilet system of claim 9, wherein the user identification input device comprises a biometric sensor.

11. The health monitoring toilet system of claim 10, wherein the biometric sensor consists of one or more of the following list: a fingerprint recognition sensor, a retinal scanner, and an iris scanner.

12. The health monitoring toilet system of claim 9, wherein the user identification input device consists of one or more of the following list: smartcard scanner, radio frequency identification reader, a near field communication transaction device, and a numerical input pad.

13. The health monitoring toilet system of claim 1, wherein the machine-readable storage medium comprises a plurality of files, wherein each of the plurality of files is associated with a unique user, and wherein analyses of urine collected from each unique user is stored in the file associated with the unique user.

14. The health monitoring toilet system of claim 13, wherein the plurality of analyses is collected over a period of time, and wherein the non-transitory computer readable medium determines a trend in urine analyte concentrations over time.

15. The health monitoring toilet system of claim 14, further comprising a communication port capable of transmitting data from the controller to an external database.

16. The health monitoring toilet system of claim 15, wherein the communication port is capable of receiving data from the external database, wherein the data from the external database comprises analyses of urine uploaded from a plurality of health monitoring toilet systems, each according to claim 15.

17. The health monitoring toilet system of claim 1, wherein the non-transient computer readable medium compares an analysis of a user's urine to a reference database, the reference database comprising ranges of normal urine metabolite values.

18. The health monitoring toilet system of claim 1, wherein the non-transient computer readable medium compares an analysis of a first user's urine to a plurality of analyses of urine samples collected from a plurality of other users.

19. The health monitoring toilet system of claim 1, wherein the non-transient computer readable medium compares an analysis of a user's urine to a database of disease indicators, wherein the disease indicators comprise values of urine metabolites.

20. The health monitoring toilet system of claim 1, wherein the at least one flush water dispenser comprises a directional nozzle.
